(19) United States
(12) Patent Application Publication
(10) Pub. No.: US 2015/0164141 A1
(43) Pub. Date: Jun. 18, 2015

(54) Electronic Cigarette with Dual Atomizer Cartridge Interface

(71) Applicant: Kyle D. Newton, Colleyville, TX (US)

(72) Inventor: Kyle D. Newton, Colleyville, TX (US)

(21) Appl. No.: 14/105,926

(22) Filed: Dec. 13, 2013

(51) Int. Cl. A24F 47/00; H01M 10/42; A61L 9/03; H01M 2/10

(52) U.S. Cl. CPC A24F 47/008; H01M 2/1022; H01M 10/4257; A61L 9/03

(57) ABSTRACT

An electronic cigarette with plural of atomizing assembly sizes. A battery assembly contains a battery and control circuit, with a first mechanical connector disposed adjacent to a first end, and a second mechanical connector disposed adjacent to the first connector. The electronic cigarette also has a small atomizer assembly with a mouthpiece, an atomizer, and a liquid reservoir. A small mechanical connector is designed to engage the second mechanical connector of the battery assembly. The assembly includes a connector shroud that has a shroud mechanical connector, and that tapers from a first outer periphery, which corresponds to the elongated housing size of the battery assembly, to a second outer periphery, which corresponds to the small atomizer assembly. When assembled, the mouthpiece of the small atomizing assembly is exposed beyond the second end of the connector shroud.

### BACKGROUND OF THE INVENTION

1. Field of the Invention

The present invention relates to electronic cigarettes. More particularly, the present invention relates to an electronic cigarette that employs dual interfaces between a battery assembly and an atomizer cartridge.

2. Description of the Related Art

Electronic cigarettes have become increasingly popular in recent years. Electronic cigarettes emulate tobacco cigarettes, but without the combustion of tobacco during use. Rather than burning tobacco, a liquid is atomized within the electronic cigarette, which produces a vapor that emulates the smoke produced in a tobacco cigarette. The liquid may contain flavoring agents such as tobacco flavor, menthol, fruits, or other flavors to enhance the “smoking” experience of the electronic cigarette. The atomizing liquid is a consumable component in an electronic cigarette, while the housing, battery, and related circuitry in the electronic cigarette are suitable for repeated use. For this reason, as well as the desire of users to enjoy various flavors of atomizing liquids, suppliers of electronic cigarettes commonly divide the electronic cigarette into two components, referred to as a battery assembly and an atomizer assembly. The atomizer assembly may be a disposable item. The battery assembly houses the battery and related circuitry, while the atomizer assembly houses the atomizing liquid, the atomizer itself, and certain pneumatic passages that route the atomized vapor to a mouthpiece.

Most of the early electronic cigarette designs emulated the appearance and function of tobacco cigarettes, both in size and proportions, making them appear familiar to tobacco users. However, the relatively small proportions of these electronic cigarettes limited the capacity of the battery power reserve, and also limited the volume of atomizing liquid that could be stored in the atomizer assembly. Hereinafter, these components will be referred to as small, or smaller, in reference to similar larger components that were later developed. Unsurprisingly, electronic cigarette users sought products that overcame these limitations. Users sought electronic cigarettes with extended life of both the battery power reserve and the vaporizing liquid, which has led to larger sized battery assemblies and larger sized atomizer assemblies. However, not all users desire to utilize the larger battery assemblies and atomizer assemblies, so there remains a demand for the smaller components. This has resulted in an environment where users my utilize both larger and smaller electronic cigarette components, which are not interchangeable. Thus, it can be appreciated that there is a need in the art for an electronic cigarette design that addresses the differences in design, particularly with respect to differing sizes of electronic cigarette components.

### SUMMARY OF THE INVENTION

The need in the art is addressed by the apparatus of the present invention. The present disclosure teaches a battery assembly for an electronic cigarette for use in combination with removably connected atomizer assemblies. The battery assembly includes an elongated housing that contains a battery and a control circuit, a first mechanical connector disposed adjacent to a first end of the elongated housing, and a second mechanical connector disposed adjacent to the first mechanical connector.

In a specific embodiment of the foregoing assembly, which is useful in combination with a large atomizer assembly and a small atomizer assembly, the first mechanical connector is adapted to engage the large atomizer assembly, and the second mechanical connector is adapted to engage the small atomizer assembly. In a refinement to this embodiment, the first mechanical connector and the second mechanical connector can not be simultaneously utilized to connect the larger atomizer assembly and the smaller atomizer assembly.

In a specific embodiment, the foregoing assembly further includes a large atomizer assembly for an electronic cigarette, which includes an atomizer and a liquid reservoir therein, and that has a large mechanical connector designed to engage the first mechanical connector of the battery assembly, which are combined to provide a complete electronic cigarette assembly.

In a specific embodiment, the foregoing assembly further includes a small atomizer assembly that has a mouthpiece, and that has an atomizer and a liquid reservoir inside, and that has a small mechanical connector designed to engage the second mechanical connector of the battery assembly. In a refinement to this embodiment, the assembly further includes a connector shroud with a shroud mechanical connector at an open first end and an open second end, where the shroud is configure to taper from a first outer periphery at the open first end, which corresponds to the elongated housing size of the battery assembly, to a second outer periphery at the second end, which corresponds to the small atomizer assembly. Further, the mouthpiece of the small atomizing assembly is exposed beyond the second end of the connector shroud while the small atomizer assembly and the connector shroud are connected to the battery assembly.

In a specific embodiment of the foregoing assembly, the first mechanical connector and the second mechanical connector are concentric. In a refinement to this embodiment, the first mechanical connector is a threaded connector and the second mechanical connector is a threaded connector. In another refinement, the first mechanical connector employs external threads and the second mechanical connector employs internal threads. In yet another refinement, the first mechanical connector is a friction fit connector and the second mechanical connector is a threaded connector.

In a specific embodiment, the foregoing assembly further includes an electrical interface coupled to the control circuit, and, the electrical interface is utilized in conjunction with both of the first mechanical connector and the second mechanical connector. In another specific embodiment, the foregoing assembly further includes a pneumatic interface coupled to the elongated housing, and the pneumatic interface is utilized in conjunction with both of the first mechanical connector and the second mechanical connector.

The present disclosure also teaches an electronic cigarette assembly that functions with plural sizes of atomizing assemblies. The embodiment includes a battery assembly that has an elongated housing that contains a battery and a control circuit, and that has a first mechanical connector disposed adjacent to a first end of the elongated housing, and a second mechanical connector disposed adjacent to the first connector. The assembly also has a small atomizer assembly with a mouthpiece, an atomizer and a liquid reservoir disposed inside. It also has a small mechanical connector designed to engage the second mechanical connector of the battery assembly. In addition, the assembly includes a connector shroud that has a shroud mechanical connector at an open first end and an open second end, the shroud is configured to taper from a first outer periphery at the open first end, which corresponds to the elongated housing size of the battery assembly, to a second outer periphery at the second end, which corresponds to the small atomizer assembly. When assembled, the mouthpiece of the small atomizing assembly is exposed beyond the second end of the connector shroud.

In a specific embodiment of the foregoing assembly, the battery assembly and the small atomizer are cylindrical, and the connector shroud is conical. In another specific embodiment, the foregoing assembly further includes an electrical interface for coupling the control circuit to the atomizer, where the electrical interface is utilized in conjunction with both of the first mechanical connector and the second mechanical connector.

In a specific embodiment, the foregoing assembly further includes a pneumatic interface for coupling the elongated housing to the mouthpiece, where the pneumatic interface is utilized in conjunction with both of the first mechanical connector and the second mechanical connector. In another specific embodiment of the foregoing assembly, the first mechanical connector and the second mechanical connector are concentric.

In a specific embodiment of the foregoing assembly, the first mechanical connector is a threaded connector and the second mechanical connector is a threaded connector. In a refinement to this embodiment, the first mechanical connector employs external threads that engage the shroud connector, and the second mechanical connector employs internal threads that engage the small mechanical connector.

The present disclosure also teaches a connector shroud for an electronic cigarette assembly that includes a battery assembly, that has an elongated housing that contains a battery and a control circuit, and that has a first mechanical connector disposed adjacent to a first end of the elongated housing, and that has a second mechanical connector disposed adjacent to the first connector. The electronic cigarette also including small atomizer assembly that has an atomizer and a liquid reservoir disposed inside, and that has a small mechanical connector designed to engage the second mechanical connector of the battery assembly. The connector shroud includes a tubular shroud structure that has a shroud mechanical connector at an open first end, and an open second end. The tubular shroud structure tapers from a first outer periphery at the open first end, which corresponds to the housing of the battery assembly, to a second outer periphery at the second end, which corresponds to the small atomizer assembly.

### BRIEF DESCRIPTION OF THE DRAWINGS

FIG. 1 is a perspective view drawing of an electronic cigarette according to an illustrative embodiment of the present invention.

FIG. 2 is a perspective view drawing of an electronic cigarette according to an illustrative embodiment of the present invention.

FIG. 3 is a drawing of several electronic cigarette components according to an illustrative embodiment of the present invention.

FIG. 4 is a side view drawing of an assembled electronic cigarette according to an illustrative embodiment of the present invention.

FIG. 5 is a disassembled side view drawing of an electronic cigarette according to an illustrative embodiment of the present invention.

FIG. 6 is a side view drawing of an assembled electronic cigarette according to an illustrative embodiment of the present invention.

FIG. 7 is a disassembled side view drawing of an electronic cigarette according to an illustrative embodiment of the present invention.

FIG. 8 is a disassembled side view drawing of an electronic cigarette according to an illustrative embodiment of the present invention.

FIGS. 9A and 9B are a section view drawing and an end view drawing, respectively, an electronic cigarette battery assembly according to an illustrative embodiment of the present invention.

FIGS. 10A, 10B, and 10C are a section view drawing, and end cap drawing, and an end view drawing, respectively, of an electronic cigarette battery housing assembly according to an illustrative embodiment of the present invention.

FIGS. 11A and 11B are a section view drawing and an end view drawing, respectively, of an electronic cigarette large atomizing cartridge according to an illustrative embodiment of the present invention.

FIG. 12 is a section view drawing of a disassembled electronic cigarette atomizing cartridge and battery housing assembly according to an illustrative embodiment of the present invention.

FIG. 13 is a disassembled section view drawing of a small atomizer and a large battery assembly of an electronic cigarette according to an illustrative embodiment of the present invention.

FIG. 14 is a section view drawing of an electronic cigarette shroud according to an illustrative embodiment of the present invention.

FIG. 15 is an assembled section view drawing of a small atomizer, a large battery assembly and shroud of an electronic cigarette according to an illustrative embodiment of the present invention.

FIG. 16 is a disassembled section view drawing of a small atomizer and larger battery assembly of an electronic cigarette according to an illustrative embodiment of the present invention.

FIG. 17 is a section view drawing of an electronic cigarette shroud according to an illustrative embodiment of the present invention.

FIG. 18 is an assembled section view drawing of a small atomizer, large battery assembly and shroud of an electronic cigarette according to an illustrative embodiment of the present invention.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope hereof and additional fields in which the present invention would be of significant utility.

In considering the detailed embodiments of the present invention, it will be observed that the present invention resides primarily in combinations of steps to accomplish various methods or components to form various apparatus and systems. Accordingly, the apparatus and system components and method steps have been represented where appropriate by conventional symbols in the drawings, showing only those specific details that are pertinent to understanding the present invention so as not to obscure the disclosure with details that will be readily apparent to those of ordinary skill in the art having the benefit of the disclosures contained herein.

In this disclosure, relational terms such as first and second, top and bottom, upper and lower, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises a” does not, without more constraints, preclude the existence of additional identical elements in the process, method, article, or apparatus that comprises the element.

The illustrative embodiments presented herein disclose electronic cigarette assemblies and components that enable users to selectively arrange small and large atomizing assemblies with small and large battery assemblies. A shroud is also disclosed, which is useful to both conceal an exposed, and unused, interface connector, as well as presenting a smooth and graceful exterior surface of the assembled electronic cigarette. Note that the small battery assembly is not discussed at length in this disclosure, nor presented in the drawing figures, because it is primarily suitable for use only with the small atomizer assembly due to the mechanical connector configuration.

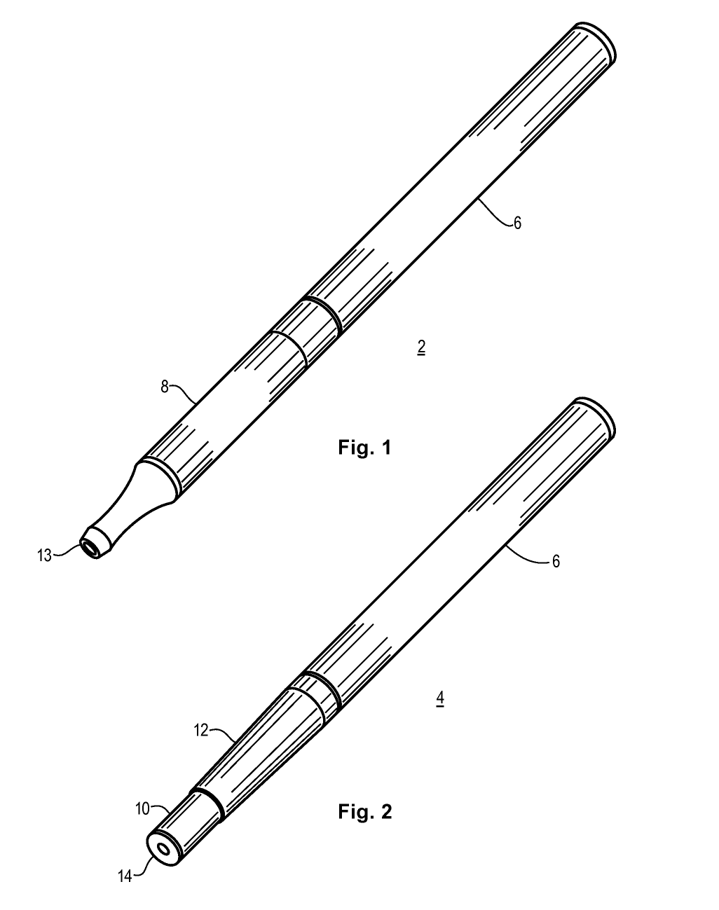

Reference is directed to FIG. 1, which is a perspective view drawing of an electronic cigarette 2 according to an illustrative embodiment of the present invention. The electronic cigarette 2 is of the large variety, and includes a large battery assembly 6 connected to a large atomizer assembly 8, which includes a mouthpiece 13 extending from one end. This is the type of electronic cigarette sought be users who preferred the increase battery capacity and increased atomizing liquid storage afforded by the use of larger components.

Reference is directed to FIG. 2, which is a perspective view drawing of an electronic cigarette 4 according to an illustrative embodiment of the present invention. This embodiment presents an assembled electronic cigarette 4 where a larger battery assembly 6 is connected to a small atomizer assembly 10, so that the user can integrate components from two differing size and capacity products. The atomizer assembly 10 includes a mouthpiece 14, which is presented at the exposed end of the small atomizer assembly 10. Note that a connector shroud 12 is also connected, and serves the functions of concealing a larger connector that is used to attach a large atomizer assembly (not shown), and also to provide a clean and graceful appearance of the assembled electronic cigarette 4.

Reference is directed to FIG. 3, which is a drawing of several electronic cigarette components according to an illustrative embodiment of the present invention. These components include larger and smaller electronic cigarettes as follows. The large battery assembly 6 includes dual connectors, including a first, large, connector 16, and a second, small, connector 18. The larger atomizer assembly 8 includes a mouthpiece 13 at one end and a larger connector 24 at the other end. Note that the large connector 24 on the large atomizer assembly 8 connects to the first connector 16 on the battery assembly 6. These two assemblies combine to yield the electronic cigarette 2 of FIG. 1. FIG. 3 also presents a small atomizer assembly 10, which includes a mouthpiece 14 at a first end and a small connector 30 at a second end. Note that the small connector 30 connects to the second connector 18 on the large battery assembly 6. This small connector 30 would also connect to a small battery, which is not illustrated or presented in this disclosure. FIG. 3 also presents a connector shroud 12, which has a shroud connector 36 at one end. The shroud connector 36 is adapted to connect to the first connector 16 on the large battery assembly 6.

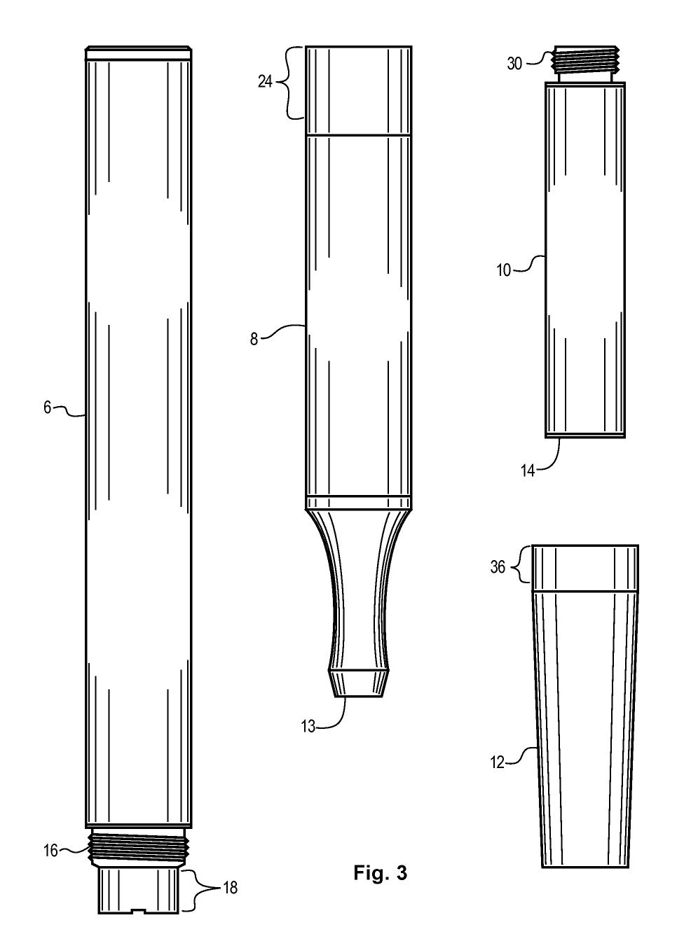

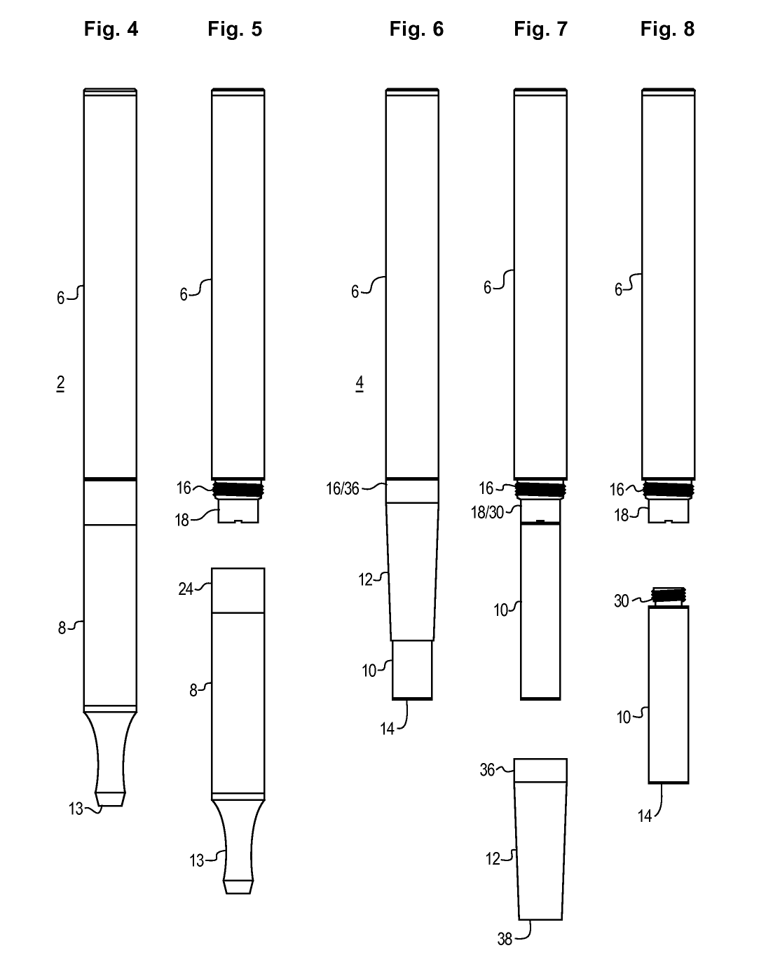

Reference is directed to FIG. 4 and FIG. 5, which are a side view drawing of an assembled larger electronic cigarette 2 and a disassembled side view drawing of a large electronic cigarette, respectively, according to an illustrative embodiment of the present invention. FIG. 4 shows the external appearance when a large battery assembly 6 is connected to a large atomizer assembly 8, including the mouthpiece 13 at the distal end of the larger atomizer assembly 8. FIG. 5 shows the components disassembled, including the large battery assembly 6 and larger atomizer assembly 8. Note that the large battery assembly 6 includes two connectors at one end, which include a first connector 16 and a second connected 18, which are different in size. The large atomizer assembly 6 includes a large connector 24 at one end, and this large connector 24 is designed to engage the first connector 16 on the large battery assembly 6. Note that in this embodiment, the second connector 18 on the larger battery assembly 6 is unused, but is concealed from view while these components are connected.

Reference is directed to FIG. 6, FIG. 7, and FIG. 8, which are a side view drawing of an assembled electronic cigarette 4, a side view drawing of a partially disassembled electronic cigarette, and a disassembled side view drawings of an electronic cigarette, respectively, according to an illustrative embodiment of the present invention. FIG. 6 illustrates a connected large battery assembly 6, small atomizer assembly 10, and connector shroud 12, which result in an assembled electronic cigarette 4. A mouthpiece 14 is presented at the distal end of the small atomizer assembly 10, and this extends beyond the end of the connector shroud 12. The connection between the large battery assembly 6 and the connector shroud 12 is accomplished with the first connector 16 on the larger battery assembly 6 and a shroud connector 36 on the connector shroud 12. This is more clearly pictured in FIG. 7, where the connector shroud 12 has been removed from the large battery assembly 6.

In FIG. 7, the exposed first connector 16 on the large battery assembly 6 can be seen. The connector shroud 12 has a shroud connector 36, which engages the first connector 16. Note that the distal end 38 of the connector shroud 12 is open to allow clearance for the small atomizer assembly 10. The small atomizer assembly 10 is connected to the second connector 18 of the large battery assembly 6 as it engages the small connector 30 on the small atomizer assembly 10. This arrangement is more plainly visible in FIG. 8, where all of these connectors 16, 18, and 30 are exposed.

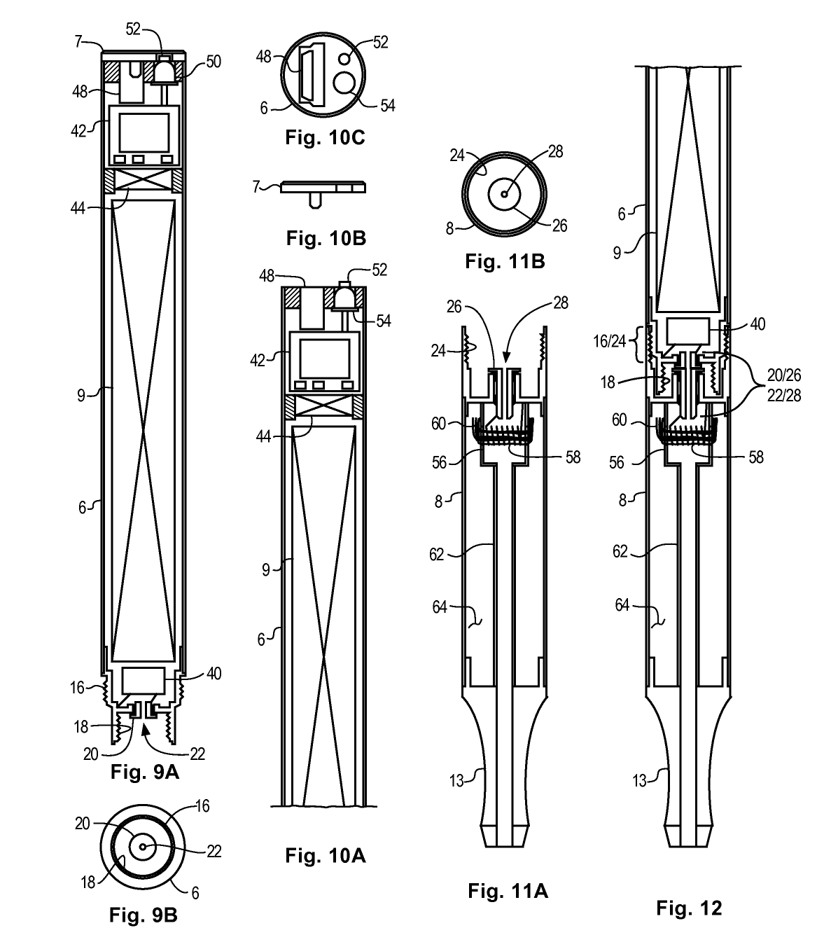

Reference is directed to FIGS. 9A and 9B, which are a section view drawing and an end view drawing, respectively, an electronic cigarette battery assembly 6 according to an illustrative embodiment of the present invention. Reference is also directed to FIGS. 10A, 10B, and 10C, which are a section view drawing, and end cap drawing, and an end view drawing, respectively, of an electronic cigarette battery assembly according to an illustrative embodiment of the present invention. These figures presents further details, including internal structure and components of the large battery assembly 6. The first connector 16 is an external machine tread at one end of the large battery assembly 6. The second connector 18 is an internal machine thread that is concentric with the first connector 16. At the center of the connector end is central electric contact terminal 20, which has a pneumatic port 22 formed there through. A circuit interface 40 connects two conductors, one to the central electrical contact 20 and one to the connectors 16, 18. In this manner, the central electrical contact terminal 20 is shared with both the small and large connectors to provide a common electrical interface. So too is the pneumatic port 22. The purpose of the pneumatic port 22 is to couple the negative pressure induced when the user draws vapor to a pneumatic sensor 44 located in the large battery assembly 6. The pneumatic sensor 44 detects the draw, which causes a control circuit 42 to activate the atomizer electric power. The electric power is provided by an internal rechargeable storage battery 9. The large battery assembly 6 also includes user interface components, which include a USB port 48, and indicator lamp 50, and a push button actuator 50. A press-on end cap 7 conceals the user interface components when they are not in use.

Reference is directed to FIGS. 11A and 11B, which are a section view drawing and an end view drawing, respectively, of an electronic cigarette large atomizing cartridge 8 according to an illustrative embodiment of the present invention. The large atomizing cartridge 8 includes a mouthpiece 13, which presents a comfortable profile for drawing vapor there from. An internal chimney 62 couples the mouthpiece 13 to an atomizing chamber 56 within the large atomizer assembly 8. An atomizer 58 is disposed within the atomizing chamber 56, and a bundle of absorbent fibers 60 enables atomizing liquid (not shown) to flow by capillary action from a liquid storage chamber 64 to the atomizer 58. The large connector 24 is an internal machine thread at the end of the large atomizer assembly 8. Central to the large connector 24 is an electrical contact terminal 26, which as a pneumatic port 28 formed there through. The atomizer 58 is a coil of nickel-chromium wire that is electrically bonded to both the electrical contact terminal 26 and the large connector 24. The pneumatic port serves as the entry point for air to enter the atomizing chamber 56, for flow out through the chimney 62 and mouthpiece 13.

Reference is directed to FIG. 12, which is a section view drawing of an assembled electronic cigarette large atomizing assembly 8 and large battery assembly 6 assembly according to an illustrative embodiment of the present invention. This figure shows the connections between these two sub-assemblies. Note that the first connector 16 of the larger battery assembly 6 and the large connector 24 of the large atomizer assembly are engaged together 16/24. This connection facilitates the electrical connection of the two central contact terminals 20/26, respectively, and also facilitates the pneumatic port coupling 22/28. Also, note that the second connector 18 of the large battery assembly 6 is unused in this connection arrangement.

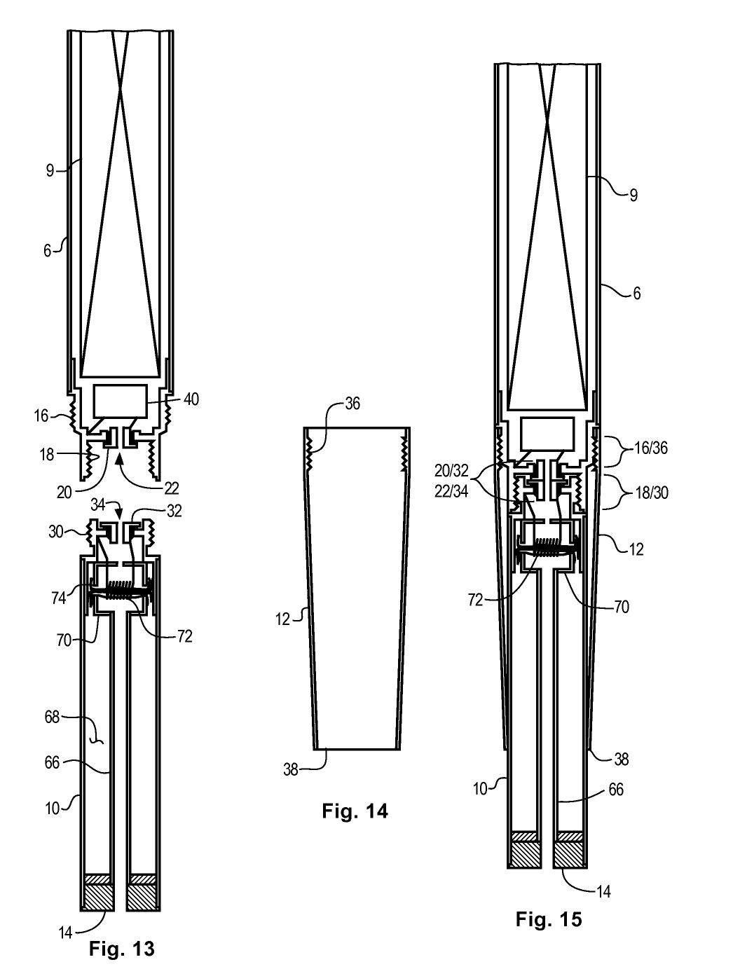

Reference is directed to FIG. 13, FIG. 14, and FIG. 15, which are a disassembled section view drawing of a small atomizer assembly 10 and large battery assembly 6, a cross section view of a connector shroud 12, and an cross section view of these components assembled together, respectively, for an electronic cigarette according to an illustrative embodiment of the present invention. The small atomizing assembly 10 includes a mouthpiece 14, which enables drawing vapor there from. An internal chimney 66 couples the mouthpiece 14 to an atomizing chamber 70 within the small atomizer assembly 10. An atomizer 72 is disposed within the atomizing chamber 70, and a bundle of absorbent fibers 74 enables atomizing liquid (not shown) to flow by capillary action from a liquid storage chamber 68 to the atomizer 72. The small connector 30 is an external machine thread at the end of the small atomizer assembly 10. Central to the small connector is an electrical contact terminal 32, which as a pneumatic port 34 formed there through. The atomizer 72 is a coil of nickel-chromium wire that is electrically bonded to both the electrical contact terminal 32 and the small connector 30. The pneumatic port 34 serves as the entry point for air to enter the atomizing chamber 70, for flow out through the chimney 66 and mouthpiece 14.

FIG. 14 is a cross section view of a connector shroud 12, which is tapered and hollow, open at both ends. A shroud connector 36, which is an internal machine thread, is located at one end of the connector shroud 12. The size of the connector shroud is selected to match the external size of the larger battery assembly 6 at the connector 36 end, and the size of the small atomizer assembly 10 at the other end. Note that in this illustrative embodiment all of the components have circular cross sections. However, various other cross sectional shapes could be employed as well, including ovals, ellipses, polygons, as well as irregular shapes.

Reference is directed to FIG. 15, which is a section view drawing of an assembled electronic cigarette small atomizing assembly 10, a connector shroud 12, and large battery assembly 6 assembly according to an illustrative embodiment of the present invention. This figure shows the connections between these three sub-assemblies. Note that the second connector 18 of the larger battery assembly 6 and the small connector 30 of the small atomizer assembly 10 are engaged together 18/30. This connection facilitates the electrical connection of the two central contact terminals 20/32, respectively, and also facilitates the pneumatic port coupling 22/34. Also, note that the first connector 16 of the large battery assembly 6 is connected to the shroud connector 36 of the connector shroud 12, labeled as 18/30.

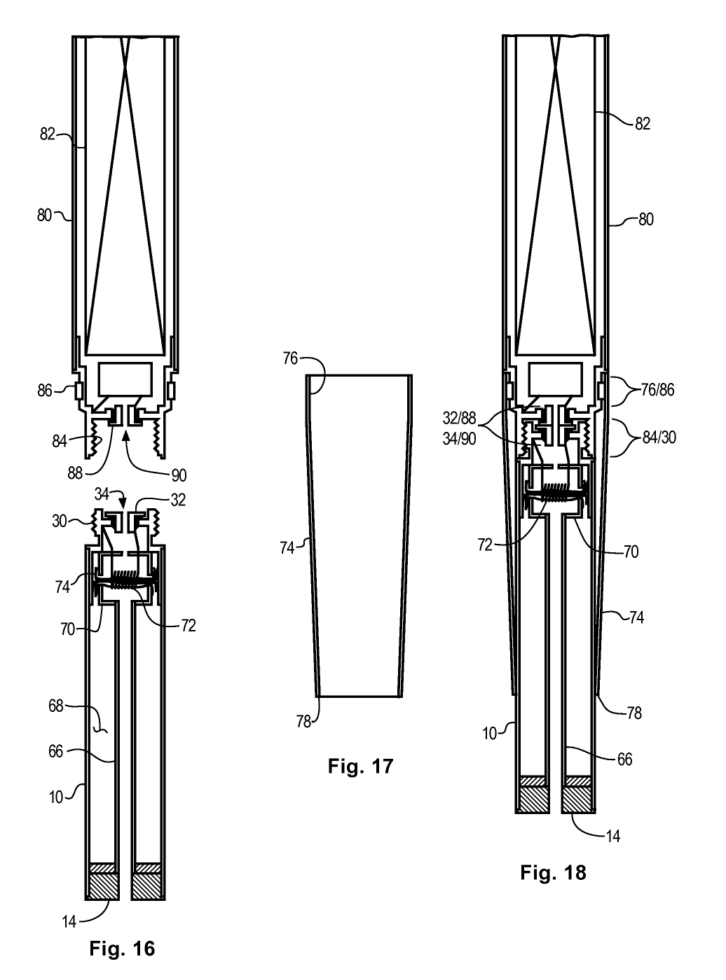

Reference is directed to FIG. 16, FIG. 17, and FIG. 18, which are a disassembled section view drawing of a small atomizer assembly 10 and large battery assembly 80, a cross section view of a connector shroud 74, and an cross section view of these components assembled together, respectively, for an electronic cigarette according to an illustrative embodiment of the present invention. In this embodiment, a friction based press-fit connection is made between the large battery assembly 80 and the connector shroud 74. In particular a polymeric band 86 is disposed to provide the first connector function. The small atomizing assembly 10 includes a mouthpiece 14, which enables drawing vapor there from. An internal chimney 66 couples the mouthpiece 14 to an atomizing chamber 70 within the small atomizer assembly 10. An atomizer 72 is disposed within the atomizing chamber 70, and a bundle of absorbent fibers 74 enables atomizing liquid (not shown) to flow by capillary action from a liquid storage chamber 68 to the atomizer 72. The small connector 30 is an external machine thread at the end of the small atomizer assembly 10. Central to the small connector is an electrical contact terminal 32, which as a pneumatic port 34 formed there through. The atomizer 72 is a coil of nickel-chromium wire that is electrically bonded to both the electrical contact terminal 32 and the small connector 30. The pneumatic port 34 serves as the entry point for air to enter the atomizing chamber 70, for flow out through the chimney 66 and mouthpiece 14.

FIG. 17 is a cross section view of a connector shroud 74, which is tapered and hollow, open at both ends. A shroud connector 74 is a smooth interior surface 76, and is located at one end of the connector shroud 74. The size of the connector shroud is selected to match the external size of the larger battery assembly 80 at the connector 76 end, and the size of the small atomizer assembly 10 at the other end.

Reference is directed to FIG. 18, which is a section view drawing of an assembled electronic cigarette small atomizing assembly 10, a connector shroud 74, and large battery assembly 80 assembly according to an illustrative embodiment of the present invention. This figure shows the connections between these three sub-assemblies. Note that the second connector 84 of the larger battery assembly 80 and the small connector 30 of the small atomizer assembly 10 are engaged together 84/30. This connection facilitates the electrical connection of the two central contact terminals 32/88, respectively, and also facilitates the pneumatic port coupling 34/90. Also, note that the first connector 86 of the large battery assembly 80 is connected to the shroud connector 76 of the connector shroud 74, labeled as 34/90. A press-fit connection is employed.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

What is claimed is:

1. A battery assembly for an electronic cigarette for use in combination with removably connected atomizer assemblies, comprising:
an elongated housing that contains a battery and a control circuit;
a first mechanical connector disposed adjacent to a first end of said elongated housing, and
a second mechanical connector disposed adjacent to said first mechanical connector.

2. The battery assembly of claim 1, for use in combination with a large atomizer assembly and a small atomizer assembly, and wherein:
said first mechanical connector is adapted to engage the large atomizer assembly, and
said second mechanical connector is adapted to engage the small atomizer assembly.

3. The battery assembly of claim 2, and wherein:
said first mechanical connector and said second mechanical connector can not be simultaneously utilized to connect the larger atomizer assembly and the smaller atomizer assembly.

4. The battery assembly of claim 1, and further comprising:
a large atomizer assembly for an electronic cigarette having an atomizer and a liquid reservoir disposed therein, and having a large mechanical connector adapted to engage said first mechanical connector of said battery assembly, thereby combined to provide a complete electronic cigarette assembly.

5. The battery assembly of claim 1, and further comprising:
a small atomizer assembly having a mouthpiece, and having an atomizer and a liquid reservoir disposed therein, and having a small mechanical connector adapted to engage said second mechanical connector of said battery assembly.

6. The battery assembly of claim 5, and further comprising:
a connector shroud having a shroud mechanical connector at an open first end and an open second end, said shroud configure to taper from a first outer periphery at said open first end, which corresponds to said elongated housing size of said battery assembly, to a second outer periphery at said second end, which corresponds to said small atomizer assembly, and wherein
said mouthpiece of said small atomizing assembly is exposed beyond said second end of said connector shroud while said small atomizer assembly and said connector shroud are engaged with said battery assembly.

7. The battery assembly of claim 1, and wherein:
said first mechanical connector and said second mechanical connector are concentric.

8. The battery assembly of claim 7, and wherein:
said first mechanical connector is a threaded connector and said second mechanical connector is a threaded connector.

9. The battery assembly of claim 8, and wherein:
said first mechanical connector employs external threads and said second mechanical connector employs internal threads.

10. The battery assembly of claim 7, and wherein:
said first mechanical connector is a friction fit connector and said second mechanical connector is a threaded connector.

11. The battery assembly of claim 1, further comprising:
an electrical interface coupled to said control circuit, and wherein
said electrical interface is utilized in conjunction with both of said first mechanical connector and said second mechanical connector.

12. The battery assembly of claim 1, further comprising:
a pneumatic interface coupled to said elongated housing, and wherein
said pneumatic interface is utilized in conjunction with both of said first mechanical connector and said second mechanical connector.

13. An electronic cigarette assembly that functions with plural sizes of atomizing assemblies, comprising:
a battery assembly having an elongated housing that contains a battery and a control circuit, and having a first mechanical connector disposed adjacent to a first end of said elongated housing, and having a second mechanical connector disposed adjacent to said first connector;
a small atomizer assembly having a mouthpiece, and having an atomizer and a liquid reservoir disposed therein, and having a small mechanical connector adapted to engage said second mechanical connector of said battery assembly;
a connector shroud having a shroud mechanical connector at an open first end and an open second end, said shroud configure to taper from a first outer periphery at said open first end, which corresponds to said elongated housing size of said battery assembly, to a second outer periphery at said second end, which corresponds to said small atomizer assembly, and wherein
said mouthpiece of said small atomizing assembly is exposed beyond said second end of said connector shroud while said small atomizer assembly and said connector shroud are engaged with said battery assembly.

14. The electronic cigarette assembly of claim 13, and wherein:
said battery assembly is cylindrical;
said small atomizer is cylindrical, and
said connector shroud is conical.

15. The electronic cigarette assembly of claim 13, and further comprising:
an electrical interface for coupling said control circuit to said atomizer, and wherein
said electrical interface is utilized in conjunction with both of said first mechanical connector and said second mechanical connector.

16. The electronic cigarette assembly of claim 13, and further comprising:
a pneumatic interface for coupling said elongated housing to said mouthpiece, and wherein
said pneumatic interface is utilized in conjunction with both of said first mechanical connector and said second mechanical connector.

17. The electronic cigarette assembly of claim 13, and wherein:
said first mechanical connector and said second mechanical connector are concentric.

18. The electronic cigarette assembly of claim 13, and wherein:
said first mechanical connector is a threaded connector and said second mechanical connector is a threaded connector.

19. The electronic cigarette assembly of claim 18, and wherein:
said first mechanical connector employs external threads that engage said shroud connector, and wherein
said second mechanical connector employs internal threads that engage said small mechanical connector.

20. A connector shroud for an electronic cigarette assembly that includes a battery assembly, having an elongated housing that contains a battery and a control circuit, and having a first mechanical connector disposed adjacent to a first end of the elongated housing, and having a second mechanical connector disposed adjacent to the first connector, the electronic cigarette also including small atomizer assembly, having an atomizer and a liquid reservoir disposed therein, and having a small mechanical connector adapted to engage the second mechanical connector of the battery assembly, said connector shroud comprising:
a tubular shroud structure;
a shroud mechanical connector at an open first end of said tubular shroud structure;
an open second end of said tubular shroud structure, and wherein
said tubular shroud structure tapers from a first outer periphery at said open first end, which corresponds to the housing of the battery assembly, to a second outer periphery at said second end, which corresponds to the small atomizer assembly.
